(19) United States
(12) Patent Application Publication
(10) Pub. No.: US 2021/0118557 A1
(43) Pub. Date: Apr. 22, 2021

(54) SYSTEM AND METHOD FOR PROVIDING MODEL-BASED PREDICTIONS OF BENEFICIARIES RECEIVING OUT-OF-NETWORK CARE

(71) Applicant: KONINKLIJKE PHILIPS N.V., EINDHOVEN (NL)

(72) Inventors: Steffen Clarence PAUWS, Eindhoven (NL); Dieter Maria Alfons VAN DE CRAEN, Balen (BE); Daniele DE MASSARI, Eindhoven (NL); Christoph Tobias WIRTH, Vellmar (DE)

(21) Appl. No.: 17/047,482

(22) PCT Filed: Apr. 17, 2019
(86) PCT No.: PCT/EP2019/059887

§ 371 Date: Oct. 14, 2020

Related U.S. Application Data
(60) Provisional application No. 62/661,968, filed on Apr. 24, 2018.

(51) Int. Cl. G16H 40/20; G06N 7/00; G06N 20/00; G16H 50/70; G16H 10/60; G16H 50/20; G06Q 10/06; G06Q 40/08; G06Q 10/10

(52) U.S. Cl. CPC G16H 40/20; G06N 7/005; G06N 20/00; G16H 50/70; G06Q 10/10; G16H 50/20; G06Q 10/06375; G06Q 40/08; G16H 10/60

(57) ABSTRACT

The present disclosure pertains to a system for providing model-based predictions of beneficiaries receiving out-of-network care. In some embodiments, the system (i) obtains, from one or more databases, a collection of information related to care utilization and expenditures for a plurality of beneficiaries; (ii) extracts, from the collection of information, information related to healthcare services rendered to the beneficiaries within a predetermined time period; (iii) provides the extracted healthcare services information to a machine learning model to train the machine learning model; (iv) obtains characteristics information related to a current beneficiary and a corresponding healthcare provider; and (v) provides, subsequent to the training of the machine learning model, the current patient and corresponding healthcare provider characteristics information to the machine learning model to predict a likelihood of a future healthcare service provided to the current beneficiary to be rendered out-of-network.

### BACKGROUND

### 1. Field

The present disclosure pertains to a system and method for providing model-based predictions of beneficiaries receiving out-of-network care.

### 2. Description of the Related Art

Care network leakage denotes the process of patients (beneficiaries, policy holders) seeking out-of-network care or being referred out-of-network by in-network healthcare providers. Network leakage may be due to a referral of an in-network professional to a provider outside of the network or the patient's own decision to seek care outside of the network. Leakage may present a significant cost burden for healthcare organization who are accountable for a population. Although automated and other computer-assisted leakage or out-of-network referral analytics systems exist, such systems may often fail to merge cost, utilization, and diagnostic claims data with socioeconomic, census, or regulatory/elective quality survey data into a holistic data set, especially given that such solutions are centered on a provider or health plan perspective which is the identification of health system leakage patterns and mitigation by outreaching to high-volume patient churn channels. These and other drawbacks exist.

### SUMMARY

Accordingly, one or more aspects of the present disclosure relate to a system for providing model-based predictions of beneficiaries receiving out-of-network care. The system comprises one or more processors configured by machine readable instructions and/or other components. The one or more hardware processors are configured to: obtain, from one or more databases, a collection of information related to care utilization of a plurality of beneficiaries; extract, from the collection of information, information related to healthcare services rendered to the beneficiaries within a predetermined time period; provide the extracted healthcare services information to a machine learning model to train the machine learning model; obtain characteristics information related to a current beneficiary and a corresponding healthcare provider; and provide, subsequent to the training of the machine learning model, the current patient and corresponding healthcare provider characteristics information to the machine learning model to predict a likelihood of a future health service provided to the current beneficiary to be rendered out-of-network.

Another aspect of the present disclosure relates to a method for providing model-based predictions of beneficiaries receiving out-of-network care with a system. The system comprises one or more processors configured by machine readable instructions and/or other components. The method comprises: obtaining, with one or more processors, a collection of information related to care utilization of a plurality of beneficiaries from one or more databases; extracting, with the one or more processors, information related to healthcare services rendered to the beneficiaries within a predetermined time period from the collection of information; providing, with the one or more processors, the extracted healthcare services information to a machine learning model to train the machine learning model; obtaining, with the one or more processors, characteristics information related to a current beneficiary and a corresponding healthcare provider; and providing, with the one or more processors, the current patient and corresponding healthcare provider characteristics information to the machine learning model subsequent to the training of the machine learning model to predict a likelihood of a future health service provided to the current beneficiary to be rendered out-of-network.

Still another aspect of present disclosure relates to a system for providing model-based predictions of beneficiaries receiving out-of-network care. The system comprises: means for obtaining a collection of information related to care utilization of a plurality of beneficiaries from one or more databases; means for extracting information related to healthcare services rendered to the beneficiaries within a predetermined time period from the collection of information; means for providing the extracted healthcare services information to a machine learning model to train the machine learning model; means for obtaining characteristics information related to a current beneficiary and a corresponding healthcare provider; and means for providing the current patient and corresponding healthcare provider characteristics information to the machine learning model subsequent to the training of the machine learning model to predict a likelihood of a future health service provided to the current beneficiary to be rendered out-of-network.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured for providing model-based predictions of beneficiaries receiving out-of-network care, in accordance with one or more embodiments.

FIG. 2 illustrates a directed acyclic graph for a Bayesian Belief Network cost estimation model for in- and out-of-network referrals, in accordance with one or more embodiments.

FIG. 3 illustrates the structure of a Bayesian Belief Network (BBN) cost estimation model, in accordance with one or more embodiments.

FIG. 4 illustrates a method for providing model-based predictions of beneficiaries receiving out-of-network care, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

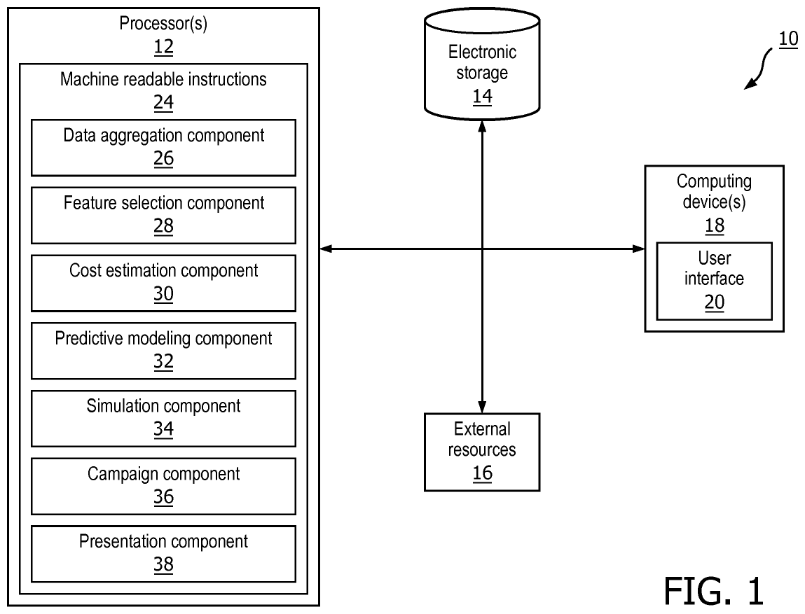

FIG. 1 is a schematic illustration of a system 10 configured for providing model-based predictions of beneficiaries receiving out-of-network care, in accordance with one or more embodiments. In some embodiments, system 10 is configured to collect and aggregate care data points from electronic health record systems, claim files, administrative databases, and publically available sources within a predetermined time period (e.g., last 12 months, year-to-date, last quarter, etc.). In some embodiments, the data is collected and aggregated at patient level which supports the creation of a care utilization profile of the beneficiaries. In some embodiments, the care utilization profile is used to understand if and to which extent covariates correlate with patients seeking care out-of-network and the amount of expenditure associated with the received out-of-network care. In some embodiments, system 10 is configured to utilize statistical or machine learning models that predict one or more of the two outcomes of interest: healthcare expenditure incurred out-of-network and the occurrence of an out-of-network care episode. In some embodiments, system 10 utilizes a model (e.g., regression model) that is trained to predict the healthcare expenditure that a patient will incur out-of-network in the next selected period given the collected covariates for the selected patients. In some embodiments, system 10 is configured to predict, via a model, whether a patient will seek care out-of-network in the next selected period. In some embodiments, responsive to a determination that a patient will seek care out-of-network given his/her current profile, system 10 is configured to (i) reach out to the patient by any available communication channel (e.g. by email) to remind him/her of all the in-network available healthcare services so that he/she will keep that in mind when planning the next visit with a specialists, (ii) alert, via a popup message or email or a flag/warning generated on a care management platform, the primary care provider to whom the patient has been assigned or the ACO medical board (if the patient has not yet been assigned) such that a professional care coordinator may review the clinical status of the patient and reach out to him/her accordingly.

In some embodiments, system 10 is configured to perform the generation of a prediction related to a likelihood of a future health service provided to a current beneficiary to be rendered out-of-network or other operations described herein via one or more prediction models. Such prediction models may include neural networks, other machine learning models, or other prediction models. As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

In some embodiments, network leakage may significantly affect systems that adopt value-based care or payment models, such as accountable care organizations (ACOs), managed care organizations (MCOs) or health managed service organizations (MSOs). In some embodiments, ACOs aim for the triple aim: improve care for the individual, improve population health, and reduce per capita costs. However, network leakage may be an impediment to ACOs for accomplishing the triple aim since once a patient leaves the ACO network, he/she may be effectively obtaining un-managed care. In some embodiments, health providers outside the network may not adhere to the same quality or cost standards. Furthermore, coordination of care among the ACO and the out-of-network providers may be challenging. Additionally, revenues that could have been generated by offering such medical services by the ACO may be counted as a loss for the ACO. Moreover, handling the fees for out-of-network services may be significantly higher than those inside the network. In some embodiments, system 10 is configured to measure, monitor, predict, and simulate care leakage from healthcare data collected in a specific geography in which a healthcare system (e.g., an ACO) operates. As such, in some embodiments, system 10 comprises processors 12, electronic storage 14, external resources 16, computing device 18, or other components.

Electronic storage 14 comprises electronic storage media that electronically stores information (e.g., collection of health information related to a plurality of beneficiaries). The electronic storage media of electronic storage 14 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., computing device 18, etc.). In some embodiments, electronic storage 14 may be located in a server together with processors 12, in a server that is part of external resources 16, and/or in other locations. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 14 may store software algorithms, information determined by processors 12, information received via processors 12 and/or graphical user interface 20 and/or other external computing systems, information received from external resources 16, and/or other information that enables system 10 to function as described herein.

External resources 16 include sources of information and/or other resources. For example, external resources 16 may include a population's electronic medical record (EMR), the population's electronic health record (EHR), or other information. In some embodiments, external resources 16 include information related to care utilization of a plurality of beneficiaries. In some embodiments, external resources 16 include sources of information such as databases, websites, etc., external entities participating with system 10 (e.g., a medical records system of a health care provider that stores medical history information of patients), one or more servers outside of system 10, and/or other sources of information. In some embodiments, external resources 16 include one or more of CMS provided CCLF (Claims and Claims Line Feeds) data sets, claims data obtained via HCUP (Healthcare Cost and Utilization Project) or ResDAC (Research Data Assistance Center), or other information. In some embodiments, external resources 16 include components that facilitate communication of information such as a network (e.g., the internet), electronic storage, equipment related to Wi-Fi technology, equipment related to Bluetooth® technology, data entry devices, sensors, scanners, and/or other resources. In some embodiments, some or all of the functionality attributed herein to external resources 16 may be provided by resources included in system 10.

Processors 12, electronic storage 14, external resources 16, computing device 18, and/or other components of system 10 may be configured to communicate with one another, via wired and/or wireless connections, via a network (e.g., a local area network and/or the internet), via cellular technology, via Wi-Fi technology, and/or via other resources. It will be appreciated that this is not intended to be limiting, and that the scope of this disclosure includes embodiments in which these components may be operatively linked via some other communication media. In some embodiments, processors 12, electronic storage 14, external resources 16, computing device 18, and/or other components of system 10 may be configured to communicate with one another according to a client/server architecture, a peer-to-peer architecture, and/or other architectures.

Computing device 18 may be configured to provide an interface between one or more users (e.g., a physician, a care coordinator, a healthcare system manager, etc.), and system 10. In some embodiments, computing device 18 is and/or is included in desktop computers, laptop computers, tablet computers, smartphones, smart wearable devices including augmented reality devices (e.g., Google Glass), wrist-worn devices (e.g., Apple Watch), and/or other computing devices associated with the one or more users. In some embodiments, computing device 18 facilitates presentation of (i) a target list of patients or physicians likely to receive or refer care out-of-network (ii) the likelihood of a future health service provided to the current beneficiary to be rendered out-of-network, (iii) out-of-network patient churn predictive covariates and a ranked list of physicians/patients generating most revenue loss caused by out-of-network utilization of health care services, (iv) required referral probabilities or maximal allowance of out-of-network referrals to comply with a defined revenue gain target, (v) insights related to the campaign, (vi) information related to the ROI of the campaign, or (vii) other information. In some embodiments, computing device 18 facilitates entry of information related to the updated referral probabilities. Accordingly, computing device 18 comprises a user interface 20. Examples of interface devices suitable for inclusion in user interface 20 include a touch screen, a keypad, touch sensitive or physical buttons, switches, a keyboard, knobs, levers, a camera, a display, speakers, a microphone, an indicator light, an audible alarm, a printer, tactile haptic feedback device, or other interface devices. The present disclosure also contemplates that computing device 18 includes a removable storage interface. In this example, information may be loaded into computing device 18 from removable storage (e.g., a smart card, a flash drive, a removable disk, etc.) that enables caregivers or other users to customize the implementation of computing device 18. Other exemplary input devices and techniques adapted for use with computing device 18 or the user interface include an RS-232 port, RF link, an IR link, a modem (telephone, cable, etc.), or other devices or techniques.

Processor 12 is configured to provide information processing capabilities in system 10. As such, processor 12 may comprise one or more of a digital processor, an analog processor, a digital circuit designed to process information, an analog circuit designed to process information, a state machine, or other mechanisms for electronically processing information. Although processor 12 is shown in FIG. 1 as a single entity, this is for illustrative purposes only. In some embodiments, processor 12 may comprise a plurality of processing units. These processing units may be physically located within the same device (e.g., a server), or processor 12 may represent processing functionality of a plurality of devices operating in coordination (e.g., one or more servers, computing device, devices that are part of external resources 16, electronic storage 14, or other devices.)

As shown in FIG. 1, processor 12 is configured via machine-readable instructions 24 to execute one or more computer program components. The computer program components may comprise one or more of a data aggregation component 26, a feature selection component 28, a cost estimation component 30, a predictive modeling component 32, a simulation component 34, a campaign component 36, a presentation component 36, or other components. Processor 12 may be configured to execute components 26, 28, 30, 32, 34, 36, or 38 by software; hardware; firmware; some combination of software, hardware, or firmware; or other mechanisms for configuring processing capabilities on processor 12.

It should be appreciated that although components 26, 28, 30, 32, 34, 36, and 36 are illustrated in FIG. 1 as being co-located within a single processing unit, in embodiments in which processor 12 comprises multiple processing units, one or more of components 26, 28, 30, 32, 34, 36, or 38 may be located remotely from the other components. The description of the functionality provided by the different components 26, 28, 30, 32, 34, 36, or 38 described below is for illustrative purposes, and is not intended to be limiting, as any of components 26, 28, 30, 32, 34, 36, or 38 may provide more or less functionality than is described. For example, one or more of components 26, 28, 30, 32, 34, 36, or 38 may be eliminated, and some or all of its functionality may be provided by other components 26, 28, 30, 32, 34, 36, or 38. As another example, processor 12 may be configured to execute one or more additional components that may perform some or all of the functionality attributed below to one of components 26, 28, 30, 32, 34, 36, or 38.

In some embodiment, the present disclosure comprises means for obtaining a collection of information related to care utilization and expenditures for a plurality of beneficiaries from one or more databases (e.g., electronic storage 14, external resources 16, etc.). In some embodiments, such means for obtaining takes the form of data aggregation component 26. In some embodiments, the healthcare utilization of the plurality of beneficiaries includes one or more of health care service or procedure items per patient (i.e., overall and out-of-network utilization (as count and as percentage of total counts), cost for health care service or procedure items per patient (i.e., overall and out-of-network spending (as monetary amount and as percentage of the total cost of care), number of visits to a provider in the selected time period per health care service or procedure items per patient, number of visits to a provider in the selected time period per health care service or procedure items per patient with a referral from the attributed primary care provider or a specialist or without a referral. In some embodiments, healthcare services include one or more of consultations, medication prescriptions, procedures (e.g., surgical procedures), therapy, or other healthcare services. In some embodiments, the collection of information is normalized and aggregated at an event-level. In some embodiments, an event includes a healthcare service rendered to a beneficiary. In some embodiments, the collection of information comprises a matrix having rows that correspond to healthcare services rendered and columns that represent one or more features, such as a set or group of service or care items, used to define the event. In some embodiments, each feature vector comprises one or more of claim-derived features, patient characteristics, provider characteristics, or other features. In some embodiments, data aggregation component 26 is configured to obtain information related to referrals from one or more scheduling systems wherein both the referring and referred providers are listed. In some embodiments, responsive to data from scheduling systems being unavailable, data aggregation component 26 is configured to utilize the concept of patient sharing (e.g., derived from claim data) as a proxy for referrals. In some embodiments, patient sharing is defined as any treatment association of providers with the patient whereas referrals require a formalized process of sending and receiving a patient for care. In some embodiment, the present disclosure comprises means for obtaining characteristics information related to a current beneficiary and a corresponding healthcare provider. In some embodiments, such means for obtaining takes the form of data aggregation component 26.

In some embodiments, claim-derived features include one or more rendered service line items as reported on the claim, a cost amount reimbursed for the service rendered as reported in claim, a label “in-network” or “out-of-network” for the service rendered, or other features. In some embodiments, responsive to the national provider identifier (NPI) of the provider (e.g., stored in the claim) being listed in the provider roster file of the healthcare system, the beneficiary receiving the service is considered “in-network”. In some embodiments, responsive to the NPI of the provider not being listed in the provider roster file of the healthcare system, the beneficiary receiving the service is considered “out-of-network”.

In some embodiments, the patient characteristics include one or more of health insurance claim number (HIC), age, gender, health insurance related information (e.g., plan type, benefits, out-of-pocket spending/deductibles/co-payments), active, inactive and currently treated diagnosis, medical history, medications, patient education level, patient satisfaction/experience, socio-economic status, type and number of procedures, overall number of services received, number of services received in-network, number of services received out-of-network, overall healthcare cost, total costs for services received out-of-network, total costs for services received in-network, or other information.

In some embodiments, the provider characteristics include one or more of healthcare system (e.g., accountable care organizations) characteristics, characteristics of in-network and out-network healthcare facilities, healthcare system catchment area characteristics at county, district, or city level, characteristic of the operating provider (wherein the operating provider is identified by the national provider identifier (NPI) listed in the claim), or other information.

In some embodiments, the healthcare system characteristics include one or more of high patient attribution turnover or fluctuation, high discontinuity of in-network care delivery, insufficient level of primary or community care, high prevalence of long-term or ambulatory care sensitive conditions (ACSCs), low Primary Care Provider (PCP) engagement, specialty deserts or specialty gaps or other information.

In some embodiments, healthcare systems do not have the authority to force patient to take in-network service. In some embodiments, healthcare systems are predominantly engaged with physicians (i.e., not patients). In some embodiments, attribution of patients to a healthcare system is determined based on Centers for Medicare & Medicaid Services (CMS) regulations. As such, patients may be categorized into attributed beneficiaries (e.g., a true customer) to the network or into assignable, potentially attributed beneficiaries for the next period or into incidental recipients of urgent care services. In some embodiments, when patient are discharged from an in-network hospital, any follow-up care (e.g., follow-up care after a planned intervention in the hospital, post-discharge care after an acute episode, etc.) may be covered by in-network providers. In some embodiments, responsive to an existence of a discontinuity in the follow-up care by the network (e.g., if the outpatient care is not well coordinated within the network), patients may be more likely to experience out-network service. In some embodiments, a primary care provider may be the first person being interfaced with when seeking care. In some embodiments, the PCP may choose and refer a patient to an in-network specialist when specialty care is needed. In some embodiments, responsive to an inadequacy of such gate keeping functionality, a patient may be more likely to receive out-of-network care. In some embodiments, hospital conditions due to complications of some long-term conditions (e.g., ambulatory care conditions including congestive heart failure, type I diabetes, hypertension, or other conditions) may be avoided if appropriate and timely outpatient or primary care is provided to patients suffering from such conditions. In some embodiments, primary care providers may not be aware which referred provider is “in-network” or “out-network” resulting into low healthcare system familiarity. In some embodiments, low primary care provider engagement may cause the PCP being or feeling disconnected from the network, providers being members of various in-network and out-network facilities, IT infrastructure and directories not being fully operational allowing PCPs to select in-network providers. In some embodiments, healthcare systems may be required to offer a whole spectrum of medical (sub-) specialties to provide full medical service to its patients. In some embodiments, such medical practices may be grouped into several categories including surgical or medical specialties, by diagnostic or therapeutic or procedure-bases methods, by place of service, by age range of patients, or by other categories. In some embodiments, responsive to a healthcare system being unable to offer connected specialties, patients who seek care from such specialties may be more likely to be referred outside the network.

In some embodiments, characteristics of in-network and out-network healthcare facilities include one or more of healthcare facility characteristics, limited access to care, Law of Roemer, low perceived quality of care, high case-mix index, or other characteristics.

In some embodiments, variation in leakage (e.g., referral to out-of-network providers) may be attributed to hospital features including ownership, volume, teaching status, staffing level, location (rural, urban), service lines or other features. In some embodiments, responsive to access to in-network providers being limited due to waiting lists or temporarily unavailability, referral to an out-network provider (who can treat or service a patient immediately) may be more likely. In some embodiments, responsive to a healthcare facility in a healthcare system having a high number of beds available, the healthcare facility may be more likely to handle a high inpatient volume and attract in-network care (i.e., based on Roemer's law stating ‘a bed built is a bed filled’ the risk of getting admitted to a hospital may be higher responsive to a higher number of beds being available in the hospital). In some embodiments, referral may be partly based on the quality of care of the referred provider or facility. In some embodiments, patients may be referred to the providers from whom they will receive the highest quality of care. In some embodiments, case-mix index (CMI) reflects the diversity and clinical complexity of patients in a catchment area or hospital. In some embodiments, CMI may be a relative value assigned to diagnosis-related groups (DRG) in claims data of beneficiaries. In some embodiments, CMI may be used to determine the allocation of resources within a DRG group by risk adjustment. In some embodiments, out-of-network referral may be attributed to CMI.

In some embodiments, healthcare system catchment area characteristics include one or more of varied (socio-) demographics, low socioeconomic class, travelability (e.g., long distance, availability of transportation means or any other geographical hurdles), or other characteristics.

In some embodiments, patient-level socio-demographic features, which can be extracted, for example, from zip-code level census data or any other commercially available data set, may include gender, age, ethnical, health literacy, social support, living arrangement, employment status, educational level distribution within an area, or other demographics. In some embodiments, the patient-level socio-demographic features may be indicative of a level of in-network care request and use (e.g., hospital (re)admission, length of stay and medical expense). In some embodiments, socioeconomic class may predict a patient's loyalty to a healthcare system (i.e., indicated by a high level of in-network care received). In some embodiments, demarcation in socioeconomic classes may indicate that patients are less informed to fully understand the benefits of receiving in-network care, whom to interface with first when needing care (e.g., the primary care provider), and hence less likely to receive in-network care. In some embodiments, patients who need to travel long distance to receive in-network care may compromise the quality of care that they will receive for reduced travel efforts. In some embodiments, geographical hurdles (e.g., passing a mountain, bridging a river, bad public transport, or other factors) to travel from a patient's place of residence to a healthcare facility to receive in-network care may cause the patient to seek out-of-network care to avoid such geographic hurdles.

In some embodiments, characteristics of a provider includes one or more of a number of attributed beneficiaries, actual patients, a total number of referrals sent in the past 12 months or other time intervals, a number of referrals sent to out-of-network providers in the past 12 months or other time intervals, a number of referrals sent to in-network providers in the past 12 months or other time intervals, a total number of referrals received from in-network providers in the past 12 months or other time intervals, a number of referrals received from out-of-network providers in the past 12 months or other time intervals, a number of referrals received from in-network providers in the past 12 months or other time intervals, a total cost reimbursed for services rendered by the selected provider, a total cost reimbursed to the providers to whom the selected provider referred a patient, a total cost reimbursed to the providers to whom the selected provider referred a patient in-network, a total cost reimbursed to the providers to whom the selected provider referred a patient out-of-network, or other characteristics.

In some embodiment, the present disclosure comprises means for extracting information related to healthcare services rendered to the beneficiaries within a predetermined time period from the collection of information. In some embodiments, such means for extracting takes the form of feature selection component 28. In some embodiments, the predetermined time period includes one year prior the event, one quarter prior the event, one month prior the event, one fiscal year prior the event, or other time periods. In some embodiments, a time lag of one week or other time lags is added to the predetermined time period to separate the end of the time period from the date of an event. For example, if an event occurred on Jun. 25, 2010, the predetermined time period is one year and the time lag is one week, the features are collected within the time window spanning from Jun. 19, 2009 to Jun. 18, 2010. In some embodiments, feature selection component 28 is configured to extract, from the collection of information, patient characteristics and provider characteristics during the predetermined time period. In some embodiments, feature selection component 28 is configured to determine a response variable (e.g., a binary variable) based on the label “in-network” or “out-of-network” for the service rendered (e.g., as reported by the claim-derived features).

In some embodiments, cost estimation component 30 is configured to determine a baseline Bayesian Belief Network (BBN) cost estimation model based on health insurance data. In some embodiments, the health insurance data includes one or more of demographic and enrolment information about each beneficiary; inpatient claims with diagnosis (Dx), medical prescriptions (Rx), date of service, reimbursement amount, and institute; outpatient claim with Dx, Rx, date of service, reimbursement amount, and institute; skilled Nursing Facility (SNF) claims, Home Health Agency (HHA) claims, carrier claims, DME supplier claims, Physician Network Data System (PNDS) containing US state-level data about the provider and service networks contracted to Health Insurers, or other information.

In some embodiments, cost estimation component 30 is configured to supplement the claims data with one or more of a list of patients who are attributed to the healthcare system through their primary care provider or other provider (i.e., patient roster data of healthcare system), publicly reported hospital readmission rates from Centers for Medicare and Medicaid Services (CMS) Hospital Compare (HC), American Hospital Association (AHA) Annual Survey database (e.g., yearly updated census of 6,500+US hospitals in 1,000 attributes on organizational structure, service lines, utilization, expenses, physicians, staffing, geography, etc.), Health Resources and Services Administration's Area Health Resource File (AHRF) containing data on health care professions, hospital, healthcare facilities, census, population and environment, Nielsen Pop-Facts containing demographic estimates and projection data, now, 5 years from now across US geography, US Census Geographical data (latitude, longitude, geo-coding, name look-up, etc.) containing data and maps of US geography in various forms, or other information.

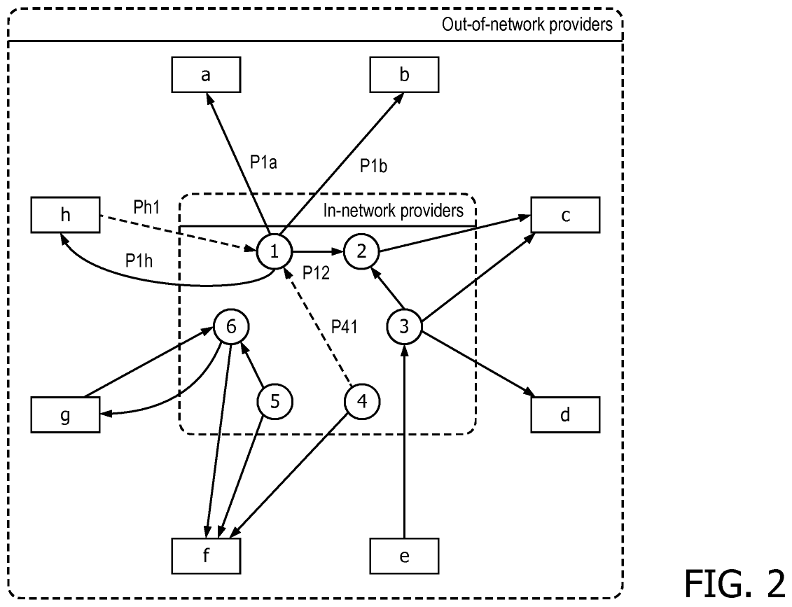

In some embodiments, the Bayesian Belief Network consists of a set of variables and a set of directed links between any two variables. In some embodiments, the link is indicated by a directional arrow leading from the cause variable to the effect variable. In some embodiments, the causal relations or links in the network are quantified by assigning conditional probabilistic values to express their strengths. In some embodiments, such conditional probabilities are evaluated using the Bayesian theory. By way of a non-limiting example, FIG. 2 illustrates a directed acyclic graph for a Bayesian Belief Network cost estimation model for out-of-network referrals, in accordance with one or more embodiments. As shown in FIG. 2, the width of the edges are proportional to the referral probability between the two connected providers/physicians. In FIG. 2, in-network providers are depicted in the inner circle and out-of-network providers are captured in the outer ring. The size of the physician nodes may scale with the total dollar amount received (e.g., for out-of-network providers) or sent out-of-network (e.g., for in-network providers).

Returning to FIG. 1, in some embodiments, data aggregation component 26 is configured to obtain health insurance claims data associated with a plurality of beneficiaries and providers (e.g., from one or more data bases associated with electronic storage 14, external resources 16, direct communication with clients, Market Scan, etc.). In some embodiments, cost estimation component 30 is configured to select baseline data by filtering the claims data (e.g., derived from health insurance claims data) based on one or more target criteria. In some embodiments, the target criteria includes one or more of episode of care, provider specialty, hospital service lines, time and/or geography, or other criteria. In some embodiments, cost estimation component 30 is configured to determine referral probabilities and cost distributions per provider/physician pair based on the selected baseline data. In some embodiments, cost estimation component 30 is configured to determine, based on the baseline claims data, (i) physician referral cost distribution baseline matrix, (ii) physician referral probabilities baseline matrix, or (iii) other information. In some embodiments, cost distributions are separable into claims sent-in-network, claims sent-out-of-network, claims received-in-network, and claims received-out-of-network. In some embodiments, cost estimation component 30 is configured to create a baseline Bayesian Belief Network (BBN) cost estimation model based on the physician referral cost distribution baseline matrix, physician referral probabilities baseline matrix, or other information.

In some embodiments, cost estimation component 30 is configured to create a trained Bayesian Belief Network (BBN) cost estimation model based on predicted referral probabilities. In some embodiments, the predicted referral probabilities are determined based on the machine learning model predictions (e.g., as described below). In some embodiments, cost estimation component 30 is configured to determine predicted referral probabilities based on (i) the previously generated probability of a future healthcare service being rendered in-network or out-of-network (e.g., as determined via the machine learning model), (ii) the baseline referral probabilities per provider/physician pair, or (iii) other information. In some embodiments, baseline in-network and out-of-network referral probabilities are extracted from referral management tools, or the referral probabilities computed from patient sharing patterns derived from claims or other sources.

In some embodiments, physician referrals are characterized by a conditional probability matrix. For example, a patient with a given condition (e.g., diagnosed by the referring physician for a required procedure for a subsequent visit) is probability weighted for a referral. In some embodiments, such a referral may be in- or out-of-network. In some embodiments, predictive modeling component 32 is configured to determine probabilities for the decision making process of physicians involved in a referral of a patient either in- or out-of-network. In some embodiments, the physician-level covariates which influences the probabilities of decision making on referrals may contain one or more of physician social circle, available list of physicians to refer to (e.g., compiled per procedure and certain stage or level per defined episodes of care), healthcare system network referral approval mechanism, patient preference communicated to physician, or other covariates.

In some embodiments, predictive modeling component 32 is configured to determine covariates influencing beneficiaries for their own decision on outmigration for service utilization out-of-network. In some embodiments, the beneficiary-level covariates or churn factors include one or more of age, gender, financial constraints, income, type of insurance (deductible, co-pay, co-insurance), willingness of out-of-pocket spending, loyalty to healthcare system and satisfaction of actually received services, perception of quality of care (e.g., as a net promotor score for in- and out-of-network care), access to care (e.g., location, availability of appointments), external information (e.g., recommendations by family and friends or health care professionals, word of mouth, published physician and hospital quality metrics or online ratings), personal preferences in care, or other covariates.

In some embodiments, data aggregation component 26 is configured to obtain updated information related to referral probabilities corresponding to the trained Bayesian Belief Network cost estimation model (e.g., for simulation of the updated information). In some embodiments, cost estimation component 30 is configured to create an updated Bayesian Belief Network (BBN) cost estimation model based on the updated referral probabilities. For example, in a simulation scenario, referral probabilities being provided as input to the trained and/or baseline Bayesian Belief Network (BBN) cost estimation model are replaced by an updated or new set of probabilities. In this example, the previous provider/physician pair may be estimated to have a baseline referral probability of p=0.50 (i.e., 50%). If this probability is assumed to change, predicted to change, or has changed to p=0.45, then the previous p-value needs to be updated to the changed p-value. As such, an updated Bayesian Belief Network (BBN) cost estimation model is created based on the updated referral probabilities.

In some embodiments, cost estimation component 30 is configured to determine the revenue gain on provider/health system level which may be simulated when probabilities for out-of-network (OON) referrals or leakage are changed. In some embodiments, referral patterns may be described by a Bayesian Belief Network (BBN) in which the nodes represent providers/physicians and the edges represent the referral probabilities between the nodes. A BBN cost model may be trained based on churn related features from a holistic data set. In some embodiments, the directional referral probabilities may be expressed or approximated by a function of churn-related features. By updating the referral probabilities, revenue gain may be simulated by comparing the updated BBN cost model with a baseline BBN.

In some embodiments, cost estimation component 30 is configured to determine a change in revenue caused by the updated referral probabilities by comparing the previously trained Bayesian Belief Network (BBN) cost estimation model with the updated Bayesian Belief Network (BBN) cost estimation model. In some embodiments, the comparison is performed via a case-based analysis in which one or more referral probabilities are provided as input to the updated Bayesian Belief Network (BBN) cost estimation model and the previously trained (or baseline) Bayesian Belief Network (BBN) cost estimation model, and the revenue generated from each model is compared with one another. For example, revenue determined by the updated Bayesian Belief Network (BBN) cost estimation model based on a referral probability matrix (pᵢⱼ) is subtracted from revenue determined by the previously trained (or baseline) Bayesian Belief Network (BBN) cost estimation model based on a referral probability matrix (\tilde{p}ᵢⱼ). In some embodiments, a regression model is generated with respect to the Bayesian Belief Network (BBN) cost estimation models' outputs.

In some embodiments, cost estimation component 30 is configured to determine, via the trained Bayesian Belief Network (BBN) cost estimation model, one or more attributes and physicians/patients causing out-of-network expenditures. In some embodiments, predicted referral probabilities may be expressed or approximated by a function of feature vector or churn covariates x:

pᵢⱼ=fᵢⱼ(x₁, . . . ,xₘ) and Σⱼpᵢⱼ=1,

wherein i denotes a physician and indices (i,j) denote a physician referral pair.

In some embodiments, cost estimation component 30 is configured to classify (e.g., in descending order) out-of-network patient churn predictive covariates and physicians/patients (e.g., as determined via the machine learning model) generating most revenue loss. In some embodiments, cost estimation component 30 is configured to identify root-causes of patient churn channels on both provider and patient level. Churn channels may depend on the structure of the health system (e.g. ACO), provider affiliations and care journeys for diseases, and treatment associations between providers.

In some embodiments, simulation component 34 is configured to obtain (e.g., via data aggregation component 26) a referral constraints matrix indicative of one or more referral probability adjustment exclusions. In some embodiments, the referral constraints matrix indicates one or more scenarios in which beneficiaries will receive care outside of the network of providers that their health insurance or plan has arranged for. For example, a beneficiary may seek out-of-network care when in need of emergency care while traveling far outside the reach of the network. As another example, a beneficiary may seek out-of-network care when the only specialist available is not part of the network.

In some embodiments, simulation component 34 is configured to determine a target revenue gain for one or more in-network physicians. For example, a 10% revenue gain, a 15% revenue gain, a 20% revenue gain, or other revenue gains may be determined (e.g., for all cardiologists within the network). In some embodiments, simulation component 34 is configured to determine, based on the referral constraints matrix, a required referral probability to meet the target revenue gain. In some embodiments, simulation component 34 is configured to determine the required referral probability by minimizing the delta of the revenue gain target to the actual output revenue gain given a Referral Constraints matrix in the following denoted as C. In some embodiments, simulation component 34 is configured to determine monetary targets rather than actual referral counts as targets. For example, for a given baseline referral probability matrix, the updated referral matrix can be approximated by solving the equation:

(P−P₀)·cost=g,

wherein g denotes the revenue gain target vector, P denotes (pᵢⱼ) referral probability matrix, P₀ denotes baseline probability matrix, cost denotes total referral cost vector, and subject to the constraints (0≤C≤P≤1), with constraints matrix C.

In some embodiments, referral probabilities may be related to actual referrals counts by multiplication with total referrals. As such, in some embodiments, simulation component 34 is configured to facilitate a provider to determine a number of referrals still remaining to meet the monetary target. For example, for a 5% revenue gain, the required out-of-network referral probability for a selected provider was simulated to be reduced from a previous value of 0.40 to at least 0.30. In this updated model scenario, the number of out-of-network referrals for this provider may then for example, need to be reduced from the baseline value of 20 to the updated value of 15, assuming that the total referral count for this provider in the baseline and updated scenario remains constant. In some embodiments, the determined required referral probability may be stored, e.g., on electronic storage 14, as the updated referral probability (described above).

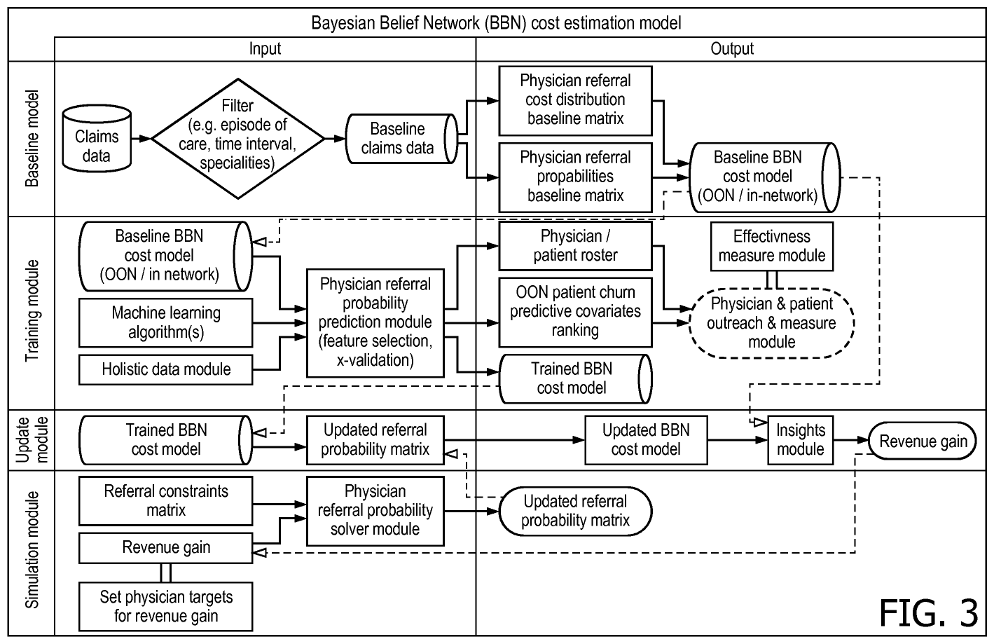

By way of a non-limiting example, FIG. 3 illustrates the structure of a Bayesian Belief Network (BBN) cost estimation model, in accordance with one or more embodiments. As shown in FIG. 3, a baseline BBN cost estimation model is created based on claims data and is provided, along with a collection of information related to health care utilization and expenditures for a plurality of beneficiaries, to a machine learning model to (i) determine out-of-network churn predictive covariates and (ii) train the baseline BBN model on predictors related to a likelihood of a future health service provided to a current beneficiary to be rendered in- or out-of-network. Furthermore, in FIG. 3, the trained BBN is updated with an updated referral probability to determine a revenue gain with the updated referral probability.

Returning to FIG. 1, in some embodiment, the present disclosure comprises means for providing the extracted healthcare services information to a machine learning model to train the machine learning model. In some embodiments, such means for providing takes the form of predictive modeling component 32. In some embodiments, predictive modeling component 32 is configured to provide the baseline BBN cost estimation model as input to the machine learning model to further train the model. In some embodiments, the machine learning model comprises a logistic regression. In some embodiments, a logistic regression fit is applied to the feature matrix and response variable. In some embodiments, the machine learning model comprises Random Forest analysis. In some embodiments, the machine learning model comprises neural networks (e.g., as described above). In some embodiments, during the training, the machine learning model infers the mapping between the input feature and the response variable based on the training dataset.

In some embodiments, a regularization technique may be used in conjunction with the machine learning model to (i) reduce the number of predictors in the model, (ii) identify important predictors, (iii) select among redundant predictors, (iv) produce shrinkage estimates with potentially lower predictive errors than ordinary least squares, (v) prevent overfitting, or (vi) enhance the prediction accuracy and interpretability of the model. As such, in the case of a logistic regression model, predictive modeling component 32 is configured to deploy a least absolute shrinkage and selection operator (LASSO) to facilitate generalization of the model. In some embodiments, LASSO may be adopted as a feature selection method to derive the subset of features carrying predictive information. In the case Random Forest analysis, features may be ranked according to variable importance during the training of the model. In some embodiments, the model may be retrained to include only the top N features. In some embodiments, N is the minimum number of top features that allows to achieve a prediction accuracy not more than 5% or other percentages lower than the prediction accuracy achieved using the full set of features. In some embodiments, responsive to the machine learning model including neural networks, L2 regularization, dimension reduction, or other regularization methods may be used. In some embodiments, responsive to the selected method not supporting categorical variables, dummy binary features may be introduced to code each possible value of a categorical feature. In some embodiments, the hyper-parameters required by the selected modelling technique are optimized using cross-validation methods.

In some embodiments, predictive modeling component 32 is configured to generate predictions related to a probability (e.g., a real number ranging from 0 to 1) of a future healthcare service to be rendered out-of-network via the machine learning model (e.g., as described above). In some embodiment, the present disclosure comprises means for providing, subsequent to the training of the machine learning model, the current patient and corresponding healthcare provider characteristics information to the machine learning model to predict a likelihood of a future health service provided to the current beneficiary to be rendered out-of-network. In some embodiments, such means for providing takes the form of predictive modeling component 32. In some embodiments, the machine learning model is configured to determine which features of the collection of information, the current patient and corresponding healthcare provider characteristics information, or other information are important. In some embodiments, predictive modeling component 32 is configured to generate per provider a roster of (attributed) patients who are predicted to receive out-of-network care.

In some embodiments, predictive modeling component 32 is configured to predict referral probabilities for a provider pair (i,j) based on:

pᵢⱼ=referrals to providerⱼ/total referrals of providerᵢ

In some embodiments, the sum of referral probabilities per physician/provider denoted i is equal to one:

Σⱼpᵢⱼ=1, for all i

In some embodiments, campaign component 36 is configured to initiate, based on the determined one or more (out-of-network churn) attributes or leakage features and target physician/patient lists, an outreach campaign. In some embodiments, a campaign includes any communicative channel by which means the behavior of providers to refer beneficiaries out-of-network will be targeted with the desired outcome to minimize out-of-network referrals. In some embodiments, a campaign may be used to influence the behavior of beneficiaries seeking care out of network with the goal to increase in-network utilization of health care services. In some embodiments, a campaign is triggered based on a target list of beneficiaries seeking care out-out of network or a target list of providers with out-of-network referral exceeding a predetermined provider-specific target. In some embodiments, campaign component 36 is configured such that the outreach campaign comprises defining, for a predetermined amount of time, a provider-specific target number of out-of-network referrals or a provider-specific target of claims dollar amounts sent out-of-network. In some embodiments, a provider target for a given time period is defined as a maximum allowance of out-of-network referrals, a maximum allowance of claims dollar amounts sent out-of-network, a maximum in-network care discontinuation level of an episode of care, or other definitions. In some embodiments, targets may be set specific to episodes of care or service lines in hospitals or provider specialties, or to geographic areas. In some embodiments, campaign component 36 is configured to classify or rank (e.g., in descending order) providers by their out-of-network referral score which measures the delta of the current referrals to the maximum allowance for a given time period.

In some embodiments, campaign component 36 is configured to provide information related to monetary impact of a campaign or model updates in terms of revenue gain. In some embodiments, campaign component 36 is configured to determine revenue gain of a campaign based on comparison of a previously defined baseline cost model (e.g., baseline BBN cost estimation model) with an updated cost model (e.g., updated BBN cost estimation model). In some embodiments, campaign component 36 is configured to determine Return of investment (ROI) with respect to a campaign. For example, campaign component 36 is configured to determine revenue gain and cost for a campaign as a function of the pool size of the campaign's recipients (e.g., number of patients or providers involved). In some embodiments, campaign component 36 is configured to maximize the ROI (revenue gain less campaign cost) for the campaign to determine the optimal pool size given one or more scenario constraints (e.g., internal or external resources and monetary or budget limitations).

In some embodiments, campaign component 36 is configured to assess the effectiveness of the campaign. In some embodiments, campaign component 36 is configured such that effectiveness is measured by a reduction in out-of-network referrals and hence revenue gained. In some embodiments, campaign component 36 is configured to track how out-of-network referrals for the selected physicians/providers are reduced with respect to a baseline period. In some embodiments, campaign component 36 is configured to assess provider performance. In some embodiments, provider assessment includes one or more of monetary assessments, clinical outcomes, provider quality, provider utilization (e.g., with respect to a specific procedure), patient satisfaction, or other assessments. In some embodiments, campaign component 36 is configured to initiate the outreach campaign based on a trigger event. In some embodiments, the trigger event includes one or more of patient satisfaction, provider utilization, or other events being below a predetermined threshold.

In some embodiments, presentation component 38 is configured to effectuate, via user interface 20, presentation of the likelihood of a future healthcare service provided to the current beneficiary to be rendered out-of-network. In some embodiments, presentation component 38 is configured to facilitate, via user interface 20, entry of information related to the updated referral probability. In some embodiments, presentation component 38 is configured to effectuate presentation of out-of-network patient churn predictive covariates and physicians/patients generating most revenue loss and services or procedures of an episode of care mostly referred out-of-network. In some embodiments, presentation component 38 is configured to effectuate presentation of the required referral probability, insights related to the campaign, information related to the ROI of the campaign, or other information. In some embodiments, presentation component 38 is configured to effectuate, via user interface 20, presentation of the monetary impact of a campaign or (BBN) model updates in terms of revenue gain or ROI.

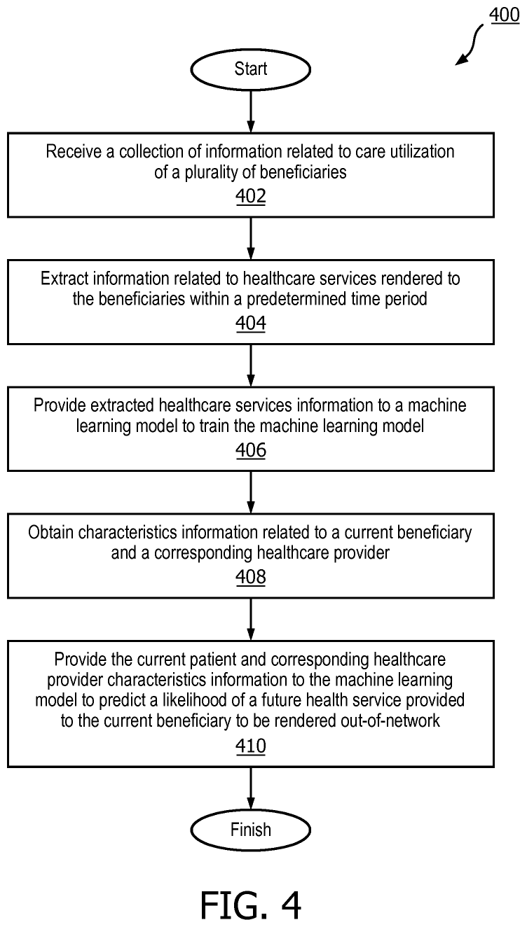

FIG. 4 illustrates a method 400 for providing model-based predictions of out-of-network care beneficiaries, in accordance with one or more embodiments. Method 400 may be performed with a system. The system comprises one or more processors, or other components. The processors are configured by machine readable instructions to execute computer program components. The computer program components include a data aggregation component, a feature selection component, a predictive modeling component, a cost estimation component, a simulation component, a campaign component, a presentation component, or other components. The operations of method 400 presented below are intended to be illustrative. In some embodiments, method 400 may be accomplished with one or more additional operations not described, or without one or more of the operations discussed. Additionally, the order in which the operations of method 400 are illustrated in FIG. 4 and described below is not intended to be limiting.

In some embodiments, method 400 may be implemented in one or more processing devices (e.g., a digital processor, an analog processor, a digital circuit designed to process information, an analog circuit designed to process information, a state machine, or other mechanisms for electronically processing information). The devices may include one or more devices executing some or all of the operations of method 400 in response to instructions stored electronically on an electronic storage medium. The processing devices may include one or more devices configured through hardware, firmware, or software to be specifically designed for execution of one or more of the operations of method 400.

At an operation 402, a collection of information related to health care utilization and expenditures for a plurality of beneficiaries is received from one or more databases. In some embodiments, operation 402 is performed by a processor component the same as or similar to data aggregation component 26 (shown in FIG. 1 and described herein).

At an operation 404, information related to healthcare services rendered to the beneficiaries within a predetermined time period is extracted from the collection of information. In some embodiments, operation 404 is performed by a processor component the same as or similar to feature selection component 28 (shown in FIG. 1 and described herein).

At an operation 406, the extracted healthcare services information is provided to a machine learning model to train the machine learning model. In some embodiments, operation 406 is performed by a processor component the same as or similar to predictive modeling component 32 (shown in FIG. 1 and described herein).

At an operation 408, characteristics information related to a current beneficiary and a corresponding healthcare provider is obtained. In some embodiments, operation 408 is performed by a processor component the same as or similar to data aggregation component 26 (shown in FIG. 1 and described herein).

At an operation 410, the current patient and corresponding healthcare provider characteristics information is provided to the machine learning model subsequent to the training of the machine learning model to predict a likelihood of a future health service provided to the current beneficiary to be rendered in- or out-of-network. In some embodiments, operation 410 is performed by a processor component the same as or similar to predictive modeling component 32 (shown in FIG. 1 and described herein).

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A system for providing model-based predictions of beneficiaries receiving out-of-network care, the system comprising:
one or more processors configured by machine-readable instructions to:
obtain, from one or more databases, a collection of information related to healthcare utilization and expenditures for a plurality of beneficiaries;
extract, from the collection of information, information related to healthcare services rendered to the beneficiaries within a predetermined time period;
provide the extracted healthcare services information to a machine learning model to train the machine learning model;
obtain characteristics information related to a current beneficiary and a corresponding healthcare provider; and
provide, subsequent to the training of the machine learning model, the current patient and corresponding healthcare provider characteristics information to the machine learning model to predict a likelihood of a future healthcare service provided to the current beneficiary to be rendered out-of-network.

2. The system of claim 1, wherein the one or more processors are configured to:
create a trained Bayesian Belief Network (BBN) cost estimation model based on referral probabilities, the referral probabilities determined based on the machine learning model predictions;
determine, via the trained Bayesian Belief Network (BBN) cost estimation model, one or more attributes and physicians/patients causing out-of-network expenditures; and
initiate, based on the determined one or more attributes and physicians/patients, an outreach campaign.

3. The system of claim 2, wherein the one or more processors are configured to:
obtain updated information related to the referral probabilities corresponding to the trained Bayesian Belief Network cost estimation model;
create an updated Bayesian Belief Network (BBN) cost estimation model based on the updated referral probabilities; and
determine a change in revenue caused by the updated referral probabilities by comparing the previously trained Bayesian Belief Network (BBN) cost estimation model with the updated Bayesian Belief Network (BBN) cost estimation model.

4. The system of claim 3, wherein the one or more processors are configured to:
obtain a referral constraints matrix indicative of one or more referral probability adjustment exclusions;
determine a target revenue gain for one or more in-network physicians; and
determine, based on the referral constraints matrix, a required referral probability to meet the target revenue gain.

5. The system of claim 2, wherein the outreach campaign comprises defining, for a predetermined amount of time, a provider-specific target number of out-of-network referrals or a provider-specific target of claims dollar amounts sent out-of-network.

6. A method for providing model-based predictions of beneficiaries receiving out-of-network care, the method comprising:
obtaining, with one or more processors, a collection of information related to care utilization and expenditures for a plurality of beneficiaries from one or more databases;
extracting, with the one or more processors, information related to healthcare services rendered to the beneficiaries within a predetermined time period from the collection of information;
providing, with the one or more processors, the extracted healthcare services information to a machine learning model to train the machine learning model;
obtaining, with the one or more processors, characteristics information related to a current beneficiary and a corresponding healthcare provider; and
providing, with the one or more processors, the current patient and corresponding healthcare provider characteristics information to the machine learning model subsequent to the training of the machine learning model to predict a likelihood of a future healthcare service provided to the current beneficiary to be rendered out-of-network.

7. The method of claim 6, further comprising:
creating, with the one or more processors, a trained Bayesian Belief Network (BBN) cost estimation model based on referral probabilities, the referral probabilities determined based on the machine learning model predictions;
determining, via the trained Bayesian Belief Network (BBN) cost estimation model, one or more attributes and physicians/patients causing out-of-network expenditures; and
initiating, with the one or more processors, an outreach campaign based on the determined one or more attributes and physicians/patients.

8. The method of claim 7, further comprising:
obtaining, with the one or more processors, updated information related to the referral probabilities corresponding to the trained Bayesian Belief Network cost estimation model;
creating, with the one or more processors, an updated Bayesian Belief Network (BBN) cost estimation model based on the updated referral probabilities; and
determining, with the one or more processors, a change in revenue caused by the updated referral probabilities by comparing the previously trained Bayesian Belief Network (BBN) cost estimation model with the updated Bayesian Belief Network (BBN) cost estimation model.

9. The method of claim 8, further comprising:
obtaining, with the one or more processors, a referral constraints matrix indicative of one or more referral probability adjustment exclusions;
determining, with the one or more processors, a target revenue gain for one or more in-network physicians; and
determining, with the one or more processors, a required referral probability to meet the target revenue gain based on the referral constraints matrix.

10. The method of claim 7, wherein the outreach campaign comprises defining, for a predetermined amount of time, a provider-specific target number of out-of-network referrals or a provider-specific target of claims dollar amounts sent out-of-network.

11. A system for providing model-based predictions of beneficiaries receiving out-of-network care, the system comprising:
means for obtaining a collection of information related to care utilization and expenditures for a plurality of beneficiaries from one or more databases;
means for extracting information related to healthcare services rendered to the beneficiaries within a predetermined time period from the collection of information;
means for providing the extracted healthcare services to a machine learning model to train the machine learning model;
means for obtaining characteristics information related to a current beneficiary and a corresponding healthcare provider; and
means for providing the current patient and corresponding healthcare provider characteristics information to the machine learning model subsequent to the training of the machine learning model to predict a likelihood of a future healthcare service provided to the current beneficiary to be rendered out-of-network.

12. The method of claim 11, further comprising:
means for creating a trained Bayesian Belief Network (BBN) cost estimation model based on referral probabilities, the referral probabilities determined based on the machine learning model predictions;
means for determining, via the trained Bayesian Belief Network (BBN) cost estimation model, one or more attributes and physicians/patients causing out-of-network expenditures; and
means for initiating an outreach campaign based on the determined one or more attributes and physicians/patients.

13. The method of claim 12, further comprising:
means for obtaining updated information related to the referral probabilities corresponding to the trained Bayesian Belief Network cost estimation model;
means for creating an updated Bayesian Belief Network (BBN) cost estimation model based on the updated referral probabilities; and
means for determining a change in revenue caused by the updated referral probabilities by comparing the previously trained Bayesian Belief Network (BBN) cost estimation model with the updated Bayesian Belief Network (BBN) cost estimation model.

14. The method of claim 13, further comprising:
means for obtaining a referral constraints matrix indicative of one or more referral probability adjustment exclusions;
means for determining a target revenue gain for one or more in-network physicians; and
means for determining a required referral probability to meet the target revenue gain based on the referral constraints matrix.

15. The method of claim 12, wherein the outreach campaign comprises means for defining, for a predetermined amount of time, a provider-specific target number of out-of-network referrals or a provider-specific target of claims dollar amounts sent out-of-network.
